(19) United States
(12) Patent Application Publication
(10) Pub. No.: US 2018/0098713 A1
(43) Pub. Date: Apr. 12, 2018

(54) HADRON THERAPY DEVICE AND MRI DEVICE HAVING MAGNETIC FIELD CORRECTING MEANS

(71) Applicant: Ion Beam Applications S.A., Louvain-Ia-Neuve (BE)

(72) Inventors: Eric FORTON, Nil-Pierreux (BE); Sébastien HENROTIN, Watermael-Boitsfort (BE)

(73) Assignee: Ion Beam Applications S.A.

(21) Appl. No.: 15/727,444

(22) Filed: Oct. 6, 2017

(30) Foreign Application Priority Data
Oct. 7, 2016 (EP) 16192761.1

(51) Int. Cl. A61B 5/055; A61B 5/00; A61N 5/10

(52) U.S. Cl. CPC A61B 5/055; A61B 5/0035; A61N 2005/1055; A61N 2005/1087; A61N 5/1039

(57) ABSTRACT

The present disclosure relates to a medical apparatus including a magnetic resonance imaging (MRI) system for acquiring magnetic resonance (MR) data from an imaging volume. In one implementation, the MRI system may include a particle beam apparatus having a particle beam line for producing, directing and managing a particle beam of charged particles, a magnetic correction device for applying a magnetic correction to a magnetic field perturbation within the imaging volume, and a controller for controlling the magnetic correction device. The controller may be configured to provide a spatially optimized magnetic correction within a restricted volume of the imaging volume during a spatially optimized magnetic correction period posterior to an excitation period during which a selected slice from the imaging volume is excited.

### CROSS-REFERENCE TO RELATED APPLICATIONS

This application claims the benefit of priority to European Application No. 16192761.1, filed Oct. 7, 2016, the contents of which are incorporated herein by reference.

### TECHNICAL FIELD

The present disclosure relates to a medical apparatus combining a hadron therapy device with a magnetic resonance imaging device. The present disclosure also relates to a method for correcting the magnetic perturbation within the imaging volume of a magnetic resonance imaging device.

### BACKGROUND

PCT Pat. publication No. WO 2015/197475 generally relates to a medical apparatus combining a MRI device and charged particle beam apparatus for the real-time magnetic resonance imaging during the delivery of the charged particles beam. For charged particle beam therapy, real-time magnetic resonance imaging (MRI) during the delivery of the charged particle beam is generally challenging because of the strong magnetic fields associated with MRI and strong magnetic fields used for directing the particle beam to the target zone. Hence, superposition of and interaction between the magnetic fields may cause severe problems, in particular for MRI. The disclosed apparatus may include an active compensation coil and a control unit, that may attempt to cancel the stray field due to the bending magnet of the beam apparatus perturbating the longitudinal component of the MRI magnetic field within the MRI imaging volume. A sufficient correction inside the sample may, however, not be easily obtained, and the current flowing in the active coils may be computed using a coil optimization program with a stream function method, which does not generally yield a solution to compensate the stray field inside the MRI imaging volume with high accuracy.

### SUMMARY

According to a first aspect, the present disclosure includes a medical apparatus that may comprise:
a magnetic resonance imaging (MRI) system for acquiring magnetic resonance data from an imaging volume, wherein the magnetic resonance imaging system comprises:
   a main magnetic unit for generating a uniform magnetic field within the imaging volume,
   gradient coils and a RF unit for generating MRI pulse sequences, said MRI pulse sequences comprising an excitation step for exciting the spin of the nuclei of the excitable atoms in a selected slice of the imaging volume during an excitation period, said MRI pulse sequences also comprising spatial encoding steps of the excited atoms during spatial encoding periods;
   antennas for receiving RF signals emitted by excited atoms;
a particle beam apparatus having a particle beam line for producing, directing and managing a particle beam of charged particles,
a magnetic correction device for applying a magnetic correction to a magnetic field perturbation within the imaging volume, and
a controller for controlling said magnetic correction device,
   wherein said controller may be configured to provide a spatially optimized magnetic correction within a restricted volume of said imaging volume during a spatially optimized magnetic correction period, said spatially optimized magnetic correction period being posterior to said excitation period and including at least one spatial encoding period of the pulse sequence, said restricted volume comprising said selected slice.

By including at least one spatial encoding period of the pulse sequence, said spatially optimized magnetic correction period may be applied during the entire duration of said spatial encoding period.

In one embodiment, the restricted volume of said imaging volume may be equal to the selected slice.

In another embodiment, the restricted volume of said imaging volume may comprise the selected slice and a buffer zone around said selected slice.

In one embodiment, the controller may be configured to provide an initial magnetic correction within the whole imaging volume during a time period preceding said spatially optimized magnetic correction period and including said excitation period. In this embodiment, the spatially optimized magnetic correction may correspond to an enhancement within said restricted volume of the initial magnetic correction.

In one embodiment, said particle beam apparatus may comprise at least one bending magnet and at least one scanning magnet for directing the particle beam to an irradiation volume within the imaging volume, said at least one bending magnet and at least one scanning magnet being able to generate an adjustable magnetic field, said controller being configured to apply said magnetic correction according to the set point information of said at least one bending magnet and at least one scanning magnet.

In one embodiment, said particle beam apparatus may comprise a gantry configured for rotating around a rotational axis, wherein the gantry may comprise said at least one bending magnet, said controller being configured to adapt the magnetic correction according to the rotation angle of the gantry.

In one embodiment, said controller may be configured to control the particle beam apparatus, the MRI system and the magnetic correction device, said controller being able to receive in input the hadron therapy treatment plan and the MRI imaging treatment plan, said controller coordinating the execution of MRI pulse sequences, the delivery of the particle beam of charged particles and the magnetic correction applied by the magnetic correction device.

In one embodiment, said magnetic correction device may comprise active coils, said active coils being arranged to allow a magnetic correction of the longitudinal and both transversal components of the magnetic field perturbation.

According to a second aspect, the present disclosure relates to a method for correcting the magnetic perturbation within the imaging volume of a MRI apparatus. Said MRI apparatus may comprise:
a main magnetic unit for generating a uniform magnetic field within the imaging volume,
gradient coils and a RF unit for generating MRI pulse sequences, said MRI pulse sequences comprising an excitation step for exciting the spin of the nuclei of the excitable atoms in a selected slice of the imaging volume during an excitation period, said MRI pulse sequences also comprising spatial encoding steps of the excited atoms during spatial encoding periods;
antennas for receiving RF signals emitted by excited atoms; and
   said method may comprise providing a spatially optimized magnetic correction within a restricted volume of said imaging volume during a spatially optimized magnetic correction period, said spatially optimized magnetic correction period being posterior to said excitation period and including at least one spatial encoding period of the pulse sequence, said restricted volume comprising said selected slice.

### SHORT DESCRIPTION OF THE DRAWINGS

These and further aspects of the present disclosure will be explained in greater detail by way of example and with reference to the accompanying drawings in which:

FIG. 1A schematically shows an example embodiment of a medical apparatus comprising a hadron therapy device coupled to a MRI, according to the present disclosure.

FIG. 1B schematically shows another example embodiment of a medical apparatus comprising a hadron therapy device coupled to a MRI, according to the present disclosure.

FIG. 2A schematically shows a selection of an imaging slice in an MRI, according to an example embodiment of the present disclosure.

FIG. 2B schematically shows a creation of phase gradients and frequency gradients during imaging of the slice of FIG. 2A, according to an example embodiment of the present disclosure.

FIG. 3 schematically shows an example MRI pulse sequence with a spatially optimized magnetic correction applied, according to an example embodiment of the present disclosure.

FIG. 4 schematically represents an example control unit, according to an example embodiment of the present disclosure.

FIG. 5 schematically represents another example control unit, according to another example embodiment of the present disclosure.

FIG. 6A illustrates an example active coil used as a magnetic correction device, according to an example embodiment of the present disclosure.

FIG. 6B illustrates another example active coils used as a magnetic correction device, according to an example embodiment of the present disclosure

FIG. 6C illustrates flowing directions for the current in the current loops from the example of FIG. 6A.

FIG. 7 represents an embodiment of a magnetic correction device comprising a passive ferromagnetic counterweight, according to an example embodiment of the present disclosure.

The figures are not drawn to scale. Generally, identical components are denoted by the same reference numerals in the figures.

### DETAILED DESCRIPTION

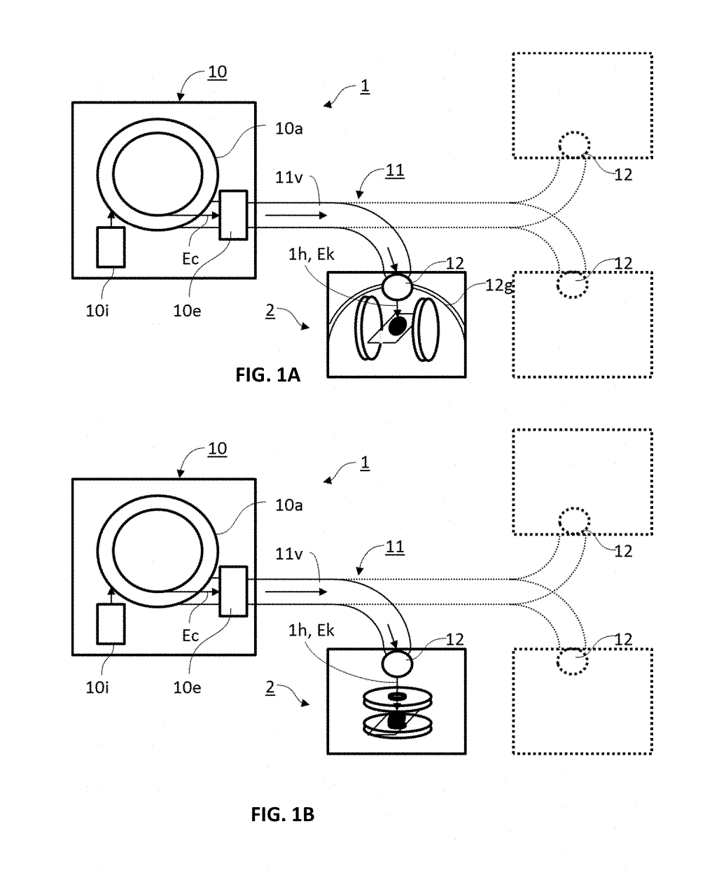

FIGS. 1A and 1B illustrate two examples of a medical apparatus comprising a hadron therapy device 1 coupled to a magnetic resonance imaging device (MRI) 2 according to embodiments of the present disclosure. A hadron therapy device and a MRI and the combination of the two are described in greater detail in the following description.

Hadron therapy device

Hadron therapy is a form of external beam radiotherapy using beams 1h of energetic hadrons. A hadron beam may be directed towards a target spot in a target tissue of a subject of interest. Target tissues of a subject of interest may include cancerous cells forming a tumour. During a hadron therapy session, a hadron beam of initial energy, Ek, with k=0 or 1, may irradiate one or more target spots within the target tissue, such as a tumour, and may destroy the cancerous cells included in the irradiated target spots, thus reducing the size of the treated tumour by necrosis of the irradiated tissues.

A hadron is a composite particle made of quarks held together by strong nuclear forces. Typical examples of hadrons include protons, neutrons, pions, heavy ions, such as carbon ions, or the like. In hadron therapy, electrically charged hadrons are generally used. For example, the hadron may be a proton, and the corresponding hadron therapy may be referred to as proton therapy. In the following, unless otherwise indicated, any reference to a proton beam or proton therapy applies to a hadron beam and/or hadron therapy in general.

As depicted in FIGS. 1A and 1B, hadron therapy device 1 generally comprises a hadron source 10, a beam transport line 11, and a beam delivery system 12. Charged hadrons may be generated from an injection system 10i, and may be accelerated in a particle accelerator 10a to build up energy. Suitable accelerators include for example, a cyclotron, a synchro-cyclotron, a synchrotron, a laser accelerator, or the like. For example, a (synchro-)cyclotron may accelerate charged hadron particles from a central area of the (synchro-)cyclotron along an outward spiral path until they reach the desired output energy, Ec, whence they may be extracted from the (synchro-)cyclotron. Said output energy, Ec, reached by a hadron beam when extracted from the (synchro-)cyclotron may typically be between 60 MeV and 350 MeV, e.g., between 210 MeV and 250 MeV. The output energy, Ec, may be or may not be the initial energy, Ek, of the hadron beam used during a therapy session; Ek may be equal to or lower than Ec, Ek≤Ec. An example of a suitable hadron therapy device includes, but is not limited to, a device described in U.S. Pat. No. 8,410,730 or in published U.S. Pat. publication No. 2012/0160996, the entire disclosures of which are incorporated herein by reference as representative of suitable hadron beam therapy devices used in the present invention.

The energy of a hadron beam extracted from a (synchro-)cyclotron may be decreased by energy selection means 10e, such as energy degraders, positioned along the beam path downstream of the (synchro-)cyclotron, which may decrease the output energy, Ec, down to any value of Ek, including down to 0 MeV. As discussed supra, the position of the Bragg peak along a hadron beam path traversing specific tissues may depend on the initial energy, Ek, of the hadron beam. By selecting the initial energy, Ek, of a hadron beam intersecting a target spot 40s located within a target tissue, the position of the Bragg peak may be controlled to correspond to the position of the target spot.

As illustrated in FIGS. 1A and 1B, downstream of the hadron source, a hadron beam of initial energy, Ek, may be directed to the beam delivery system 12 through a beam transport line 11. The beam transport line may comprise one or more vacuum ducts, 11v, and/or a plurality of magnets for controlling the direction of the hadron beam and/or for focusing the hadron beam, e.g., including the so-called bending magnet and scanning magnets. The beam transport line may also be adapted for distributing and/or selectively directing the hadron beam from a single hadron source 10 to a plurality of beam delivery systems for treating several patients in parallel.

The beam delivery system 12 may further comprise a nozzle for orienting a hadron beam 1h along a beam path. The nozzle may be fixed or mobile. Mobile nozzles are generally mounted on a gantry 12g. A gantry may be used for varying the orientation of the hadron outlet about a circle centred on an isocentre and normal to an axis, Z, which is generally horizontal. In supine hadron treatment devices, the horizontal axis, Z, may be selected parallel to a patient lying on a couch (i.e., the head and feet of the patient are aligned along the horizontal axis, Z). The nozzle and the isocentre may define a path axis, Xp, whose angular orientation may depend on the angular position of the nozzle in the gantry. By using magnets positioned adjacent to the nozzle, e.g., the so-called scanning magnets, the beam path of a hadron beam 1h may be deviated with respect to the path axis, Xp, within a cone centred on the path axis and having the nozzle as apex. Accordingly, a volume of target tissue centred on the isocentre may be treated by the hadron beam without changing the position of the nozzle within the gantry. The same may apply to fixed nozzles with the difference that the angular position of the path axis may be fixed.

### Magnetic Resonance Imaging Device

A magnetic resonance imaging device 2 (MRI) implements a medical imaging technique based on the interactions of excitable atoms present in an organic tissue of a subject of interest with electromagnetic fields. When placed in a strong main magnetic field, B0, the spins of the nuclei of said excitable atoms may precess around an axis aligned with the main magnetic field, B0, resulting in a net polarization at rest that is parallel to the main magnetic field, B0. The application of a pulse of radio frequency (RF) exciting magnetic field, B1, at the frequency of resonance, fL, called the Larmor frequency, of the excitable atoms in said main magnetic field, B0, may excite said atoms by tipping the net polarization vector sideways (e.g., with a so-called 90° pulse, B1-90) or to angles greater than 90° and may even reverse it at 180° (with a so-called 180° pulse, B1-180). When the RF electromagnetic pulse is turned off, the spins of the nuclei of the excitable atoms may return progressively to an equilibrium state yielding the net polarization at rest. During relaxation, the transverse vector component of the spins may produce an oscillating magnetic field inducing a signal which may be collected by antennas 2a located in close proximity to the anatomy under examination.

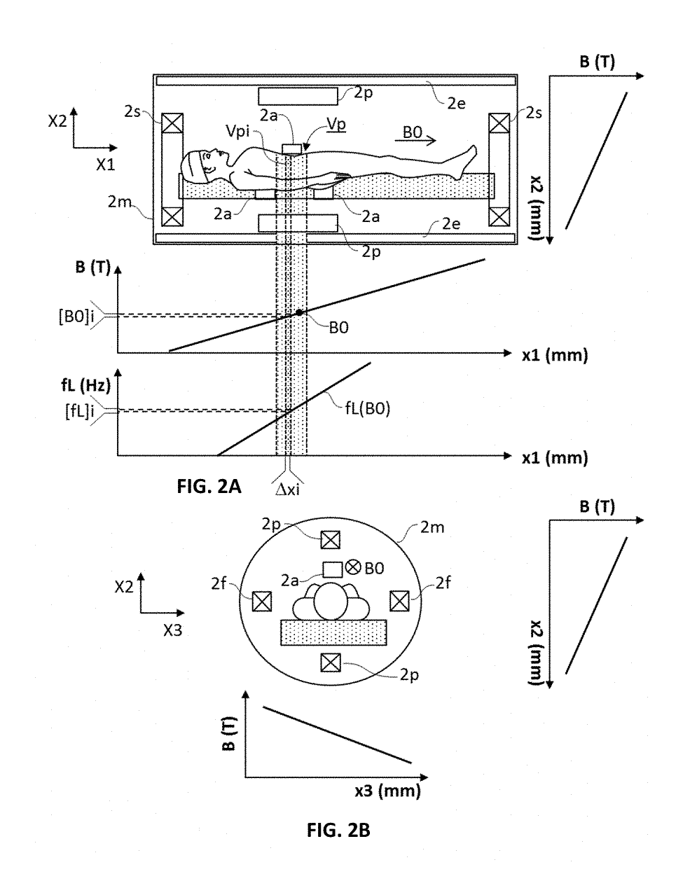

As shown in FIG. 2A, an MRI 2 may comprise a main magnet unit 2m for creating a uniform main magnetic field, B0; radiofrequency (RF) excitation coils 2e for creating the RF-exciting magnetic field, B1; X1-, X2-, and X3-gradient coils, 2s, 2p, 2f, for creating magnetic gradients along the first, second, and third directions X1, X2, and X3, respectively; and antennas 2a, for receiving RF-signals emitted by excited atoms as they relax from their excited state back to their rest state. The main magnet may produce the main magnetic field, B0, and may be a permanent magnet or an electro-magnet (e.g., a supra-conductive magnet or not). An MRI device 2 may also comprise at least one electronic controller configured to control the main magnet unit 2m, the gradient coils 2s, 2p, 2f, the excitation coils 2e and the antennas 2a. An example of a suitable MRI includes, but is not limited to, a device described in European Pat. No. 0186238, the entire disclosure of which is incorporated herein by reference.

As illustrated in FIG. 2B, an imaging slice or layer, Vpi, of thickness, Δxi, normal to the first direction, X1, may be selected by creating a magnetic field gradient along the first direction, X1. In FIG. 2B, the first direction, X1, may be parallel to the axis Z defined by the lying position of the patient, yielding slices normal to said axis Z. In other embodiments, the first direction, X1, may be any direction, e.g., transverse to the axis Z, with slices extending at an angle with respect to the patient. As shown in FIG. 2A, because the Larmor frequency, fL, of an excitable atom may depend on the magnitude of the magnetic field to which it is exposed, sending pulses of RF exciting magnetic field, B1, at a frequency range, [fL]i, may excite exclusively the excitable atoms which are exposed to a magnetic field range, [B0]i, and located in a slice or layer, Vpi, of thickness, Δxi. By varying the frequency bandwidth, [fL]i, of the pulses of RF exciting magnetic field, B1, the width, Δxi, and position of an imaging layer, Vpi, may be controlled. By repeating this operation on successive imaging layers, Vpi, an imaging volume, Vp, may be characterized and imaged.

To localize the spatial origin of the signals received by the antennas on a plane normal to the first direction, X1, magnetic gradients may be created successively along second and third directions, X2, X3, wherein X1⊥X2⊥X3, by activating the X2-, and X3-gradient coils 2p, 2f, as illustrated in the example of FIG. 2B. Said gradients may provoke a phase gradient, Δφ, and a frequency gradient, Δf, in the spins of the excited nuclei as they relax, which may allow spatial encoding of the received signals in the second and third directions, X2, X3. A two-dimensional matrix may thus be acquired, producing k-space data, and an MR image may be created by performing a two-dimensional inverse Fourier transform. Other modes of acquiring and creating an MR image may be implemented by one of ordinary skill in the art.

The MRI may be any of a closed-bore, open-bore, or wide-bore MRI type. A typical closed-bore MRI may have a magnetic strength of 1.0 T through 3.0 T with a bore diameter of the order of 60 cm. An open-bore MRI typically has two main magnet poles 2m separated by a gap for accommodating a patient in a lying position, sitting position, or any other position suitable for imaging an imaging volume, Vp. The magnetic field of an open-bore MRI may be between 0.2 T and 1.0 T. A wide-bore MRI is a kind of closed-bore MRI having a larger diameter.

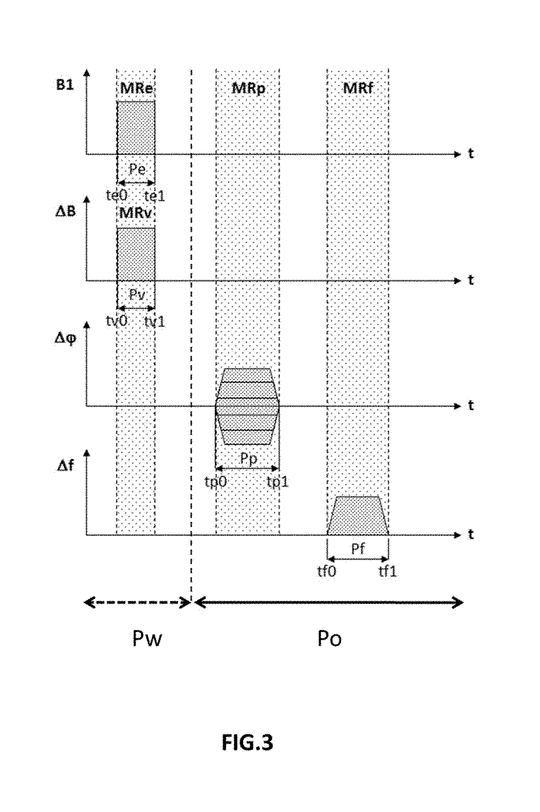

As depicted in FIG. 3, an MRI device may acquire data from a sample to image using:
an excitation step (MRe) for exciting the spin of the nuclei of the excitable atoms, which may comprise creating an oscillating electromagnetic field, B1, with the RF unit at a RF-frequency range, [fL]i, corresponding to the Larmor frequencies of the excitable atoms exposed to an ith magnetic field range, [B0]i=[Bi,0, Bi,1], during an excitation period, Pe=(te1−te0), wherein te0 is the time of the beginning of the oscillating electromagnetic field, and te1 is the end of the oscillating electromagnetic field;
a layer selection step (MRv) for selecting a layer or slice, Vpi, of the imaging volume, Vp, of thickness, Δxi, measured along the first direction, X1, e.g., by creating a magnetic field gradient along the first direction, X1, of slope dB/dx1=[B0]i/Δxi,
a phase gradient step (MRp) for localising along the second direction, X2, the origin of RF signals received by the antennas by varying a phase of the spins of the nuclei along the second direction, X2, and may comprise creating a magnetic field gradient along the second direction, X2, during a period, Pp=(tp1−tp0), wherein tp0 is the time of the beginning of the phase gradient step, and tp1 is the end of the phase gradient step, with tp0>te1; and
a frequency gradient step (MRI) for localising along the third direction, X3, the origin of RF signals received by the antennas, e.g., by varying a frequency of the spins of the nuclei along the third direction, X3,and may comprise creating a magnetic field gradient along the third direction, X3, during a period Pf=(tf1−tf0), wherein tf0 is the time of the beginning of the frequency gradient step, and tf1 is the end of the frequency gradient step, with tf0>tp1.

The steps described above is just one example of a possible implementation of a MRI pulse sequence, which may be repeated multiple times to image the successive layers of a sample. For example, a layer may have a thickness of 3 mm Other implementations, e.g., wherein the succession of the excitation step and the gradient step are different, may also be implemented. For example, the excitation step may be split into two steps, e.g., a 90° rotation of the longitudinal nuclear magnetization caused by a first RF pulse, and applying a second RF pulse after the phase gradient step to cause a 180° rotation of transverse nuclear magnetization within the excited slice, in order to form a spin echo at a subsequent time. In another implementation, the so-called MRI volumetric imaging, rather than selectively exciting a thin layer from the sample, a thick slice of about 10 cm to 20 cm may be excited by a RF pulse. The spatial encoding of the excited atoms within the thick slice may then be performed in three directions, e.g., with the help of two phase gradient steps and one frequency gradient step. In the following, a (MRI) pulse sequence will refer to a succession of RF excitation steps and gradient steps allowing one of ordinary skill to implement any appropriate MRI technique.

A medical apparatus according to one embodiment of the present disclosure may comprise:
a magnetic resonance imaging (MRI) system for acquiring magnetic resonance data from an imaging volume Vp, wherein the magnetic resonance imaging system may comprise:
   a main magnetic unit 2m for generating a uniform magnetic field within the imaging volume Vp,
   gradient coils 2s, 2p, 2f and a RF unit for generating MRI pulse sequences, said MRI pulse sequences comprising an excitation step MRe for exciting the spin of the nuclei of the excitable atoms in a selected slice Vpi of the imaging volume Vp during an excitation period Pe, said MRI pulse sequences also comprising spatial encoding steps MRp, MRf of the excited atoms during spatial encoding periods Pp, Pf; and
   antennas 2a for receiving RF signals emitted by excited atoms;
a particle beam apparatus having a particle beam line for producing a particle beam of charged particles,
a magnetic correction device for applying a magnetic correction to a magnetic field perturbation within the imaging volume Vp; and
a controller for controlling said magnetic correction device,
wherein said controller may be configured to provide a spatially optimized magnetic correction within a restricted volume of said imaging volume Vp during a spatially optimized magnetic correction period Po, said spatially optimized magnetic correction period Po being posterior to said excitation period Pe and including at least one spatial encoding period Pp, Pf of the pulse sequence, said restricted volume comprising said selected slice Vpi.

As explained above, the integration of an MRI system and a particle beam apparatus may cause problems, in particular for MRI, due to superposition and interaction between the different magnetic fields at stake. Accordingly, the magnetic correction device may restore, to some extent, a magnetic field allowing performing the selective excitation step(s) and spatial encoding step(s) to implement an MRI imaging technique. In some embodiments of the present disclosure, the controller may allow the spatial optimization of the magnetic correction during the MRI pulse sequence to yield a higher accuracy in the spatial encoding steps. Moreover, although the magnetic correction device may correct the magnetic perturbation caused by said particle beam apparatus, to the magnetic correction device may also apply a magnetic correction based on other sources of perturbation in the environment of the medical apparatus.

For example, once a slice Vpi of the imaging volume Vp has been selectively excited, the magnetic correction to perform the spatial encoding steps in this slice may be applied to a restricted volume of the imaging volume comprising the selected slice Vpi. This is because the magnitude of the magnetic field outside said selected slice Vpi usually will not harm the spatial encoding of the excited atoms within said selected slice Vpi, and consequently it may be advantageous to make full use of the magnetic correction device to apply a spatially optimized magnetic correction which may correct the magnetic field in a volume, the so-called restricted volume, which may be spatially limited compared to the full imaging volume. The spatially optimized magnetic correction may be applied during a spatially optimized magnetic correction period Po, which may be a time period including at least one spatial encoding period Pp, Pf of the pulse sequence in the selected slice Vpi. During this spatially optimized magnetic correction period Po, the magnetic correction may be applied such that the magnitude of the magnetic field in said restricted volume is comprised within the magnetic field range [B0]i=[Bi,0, Bi,1] corresponding to the Larmor frequency range [fL]i, while no correction may be required outside of said restricted volume. The dedication of the magnetic correction device to a magnetic correction within a spatially limited volume may allow reaching a greater accuracy of the correction within such restricted volume. Such restricted volume may be chosen to be equal to the selected slice Vpi or, alternatively, to be a bit larger and to provide a buffer zone between the selected slice Vpi and the rest of the imaging volume. Such buffer zone may guarantee a maximal overlap between the targeted selected slice Vpi and the restricted volume with the spatially optimized correction.

As further illustrated in FIG. 3, the spatially optimized magnetic correction period Po may be preceded by a period Pw during which a magnetic correction may be applied to the whole imaging volume Vp. Such correction may allow an accurate slice selection step MRv within the imaging volume Vp, which may imply that only the excitable atoms from said slice Vpi have their Larmor frequencies corresponding the frequency range [fL]i of the RF pulse generated by the RF unit. In this regard, the magnetic correction applied during the period Pw may be such that the magnitude of the magnetic field in said slice Vpi may be comprised within the magnetic field range [B0]i=[Bi,0, Bi,1] corresponding to the Larmor frequency range [fL]i, while at the same time the magnetic field outside of the excited selected slice Vpi may not be comprised in said range [B0]i=[Bi,0, Bi,1]. Such correction during a period Pw within the whole imaging volume Vp may, consequently, be more spatially constraining for the magnetic correction device than the spatially limited correction applied during the period Po, because during this period Pw a magnetic correction may be required outside of the excited selected slice Vpi, which may prevent its accuracy in terms of magnetic field magnitude. However, the accuracy of the magnetic correction may be more important during the spatial encoding steps than during the layer selection step MRv. If no magnetic correction is applied during the period preceding the spatially optimized magnetic correction period Po, or if the correction applied during the period Pw is not sufficient to provide an accurate selective excitation of a layer having a regular slice shape Vpi, mathematical methods may be used to reconstruct a 3D MRI image based on the acquisition of RF signals generated by said excited irregular layer, the shape of said irregular layer being deducible, for example, from the knowledge of the magnetic perturbation within said imaging volume Vp.

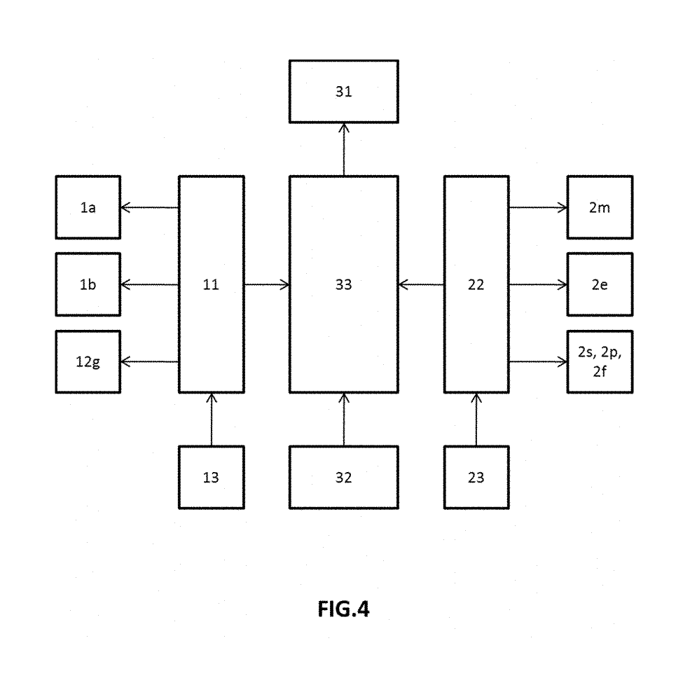

As illustrated in FIG. 4, the controller of the magnetic correction device may comprise a control unit 33 controlling active coils 31 used as magnetic correction device. The control units may receive data in input from both the control unit 11 of the hadron therapy device 1, and the control unit 22 of the MRI device 2. The control unit 11 of the hadron therapy device 1 may be responsible for the execution of a treatment plan received from the hadron therapy user input device 13 and, among other things, may exert control over the bending magnet la and the scanning magnets 1b from the beam delivery system and also on rotation angle of the gantry 12g. The set point information from said bending magnet 1a, scanning magnets 1b and gantry 12g may be delivered to the control unit 33. The control unit 22 of the MRI, on the other hand, may exert control over the units generating the MRI pulse sequences, which may include the excitation coils 2e, the main magnetic unit 2m and/or the gradient coil 2s, 2p and 2f. The set point information from the units generating the MRI pulse sequences may also be delivered by the MRI control unit 22 to the control unit 33. The control unit 33 may also receive, from an input device 32, look-up tables of values of the correcting current to apply in the active coils in dependence of the currents flowing through the bending magnet 1a and/or the scanning magnets 1b and/or of the angle of rotation of the gantry 12g. Such look-up tables may be obtained from simulations or experiments or a combination thereof and may also use of an optimization algorithm that computes the currents in the active coil minimizing the magnetic field perturbation across the MRI imaging volume. As the control unit 33 receives input data related to the MRI pulse sequences from the control unit 2, it may compute the spatially optimized magnetic correction within the restricted volume comprising selected slice Vpi and the corresponding period Po, as described above.

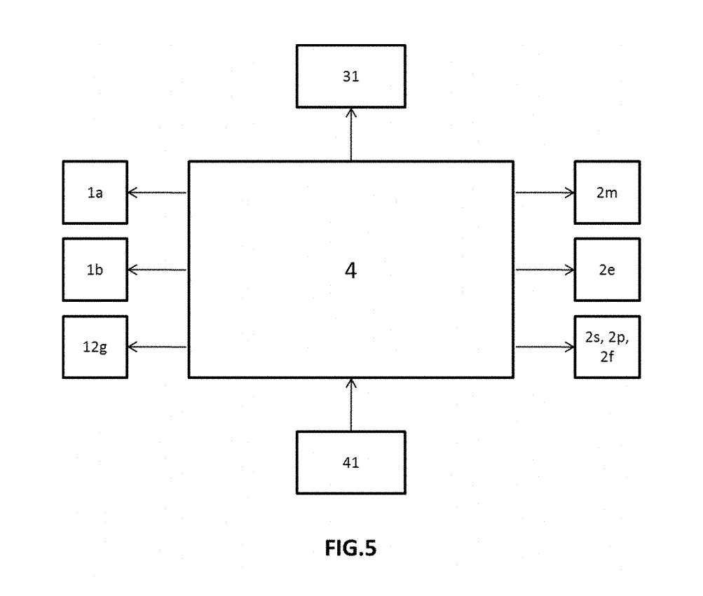

In an alternative embodiment illustrated in FIG. 5, the controller may comprise a central unit 4 controlling, at the same time, the active coils 31, the various units of the hadron therapy device 1 and those of the MRI device 2. In such an embodiment, this central unit 4 may receive, from its input device 41, a broad range of information comprising the hadron therapy treatment plan, the MRI imaging plan and/or look-up tables of the correcting current in the active coils 31 as described above, and may optimize the global imaging and/or treatment sequence and then monitor the hadron therapy treatment and/or the MRI imaging process to coordinate the successive treatment steps, MRI pulse sequence steps and/or the magnetic correction delivered by the active coils 31.

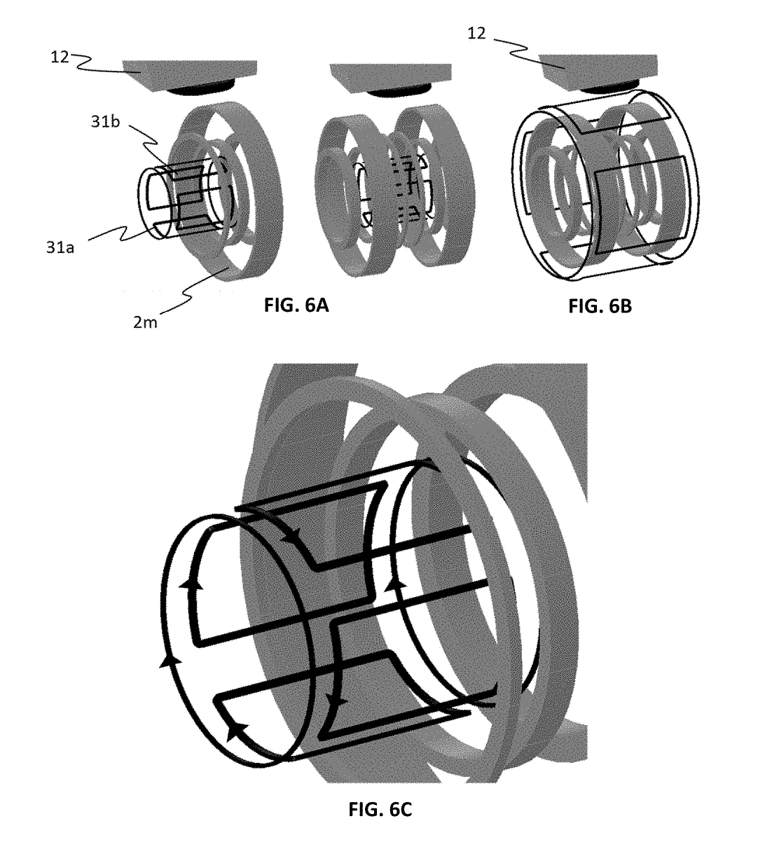

FIGS. 6A and 6B illustrate two embodiments of the active coils 31 used as a magnetic correction device. In FIG. 6A, the active coils 31 may comprise several current loops arranged in proximity to the inner surface of the MRI main magnetic unit 2m. The active coils 31 may comprise one pair of lateral current loops 31a and several pairs of longitudinal current loops 31b. The presence of multiple current loops of various geometries 31a and 31b may allow for correcting efficiently the magnetic perturbations inside the imaging volume of the MRI device. For example, such coils may allow for correcting the components of the magnetic field perturbation in the three spatial dimensions. In one embodiment, the current in each loop may be driven independently in order to maximize the degrees of freedom of the magnetic correction. In FIG. 6B, the active coils 31 may be arranged in proximity to the outer surface of the MRI main magnetic unit 2m. The end of the beam delivery system 12 may also be represented in these figures. FIG. 6C shows possible flowing directions for the current in the current loops from the example of FIG. 6A. A magnetic correction device according to the present disclosure comprise more sophisticated coil geometries and/or with also a greater number of coils, wherein the current is driven either independently or not, e.g., in order to increase the number of degrees of freedom for increasing the accuracy of the spatially optimized magnetic correction. In some embodiments, the MRI gradient coils may also be used as a magnetic correction device such that the current flowing through these coils may be chosen to compensate in some extent the magnetic perturbation generated inside the MRI imaging volume Vp, especially within excited slice Vpi.

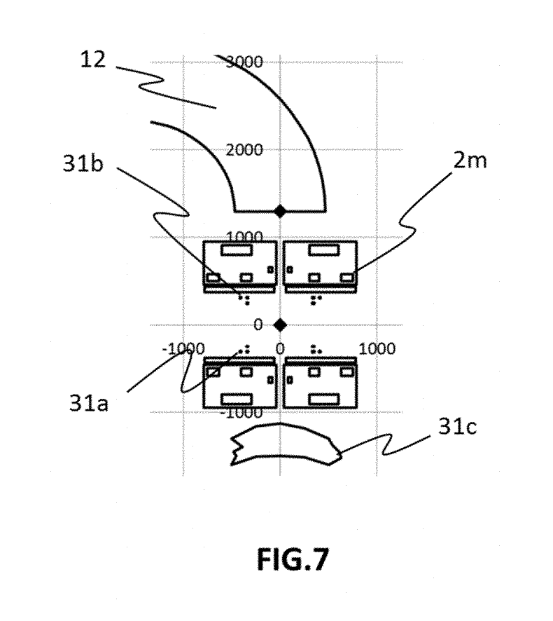

FIG. 7 illustrates an example embodiment of a passive ferromagnetic counterweight 31c used as a magnetic correction device. Passive ferromagnetic counterweight 31c may complement the active coils 31 and may compensate the magnetic perturbation generated by the metallic structure of the gantry 12g. Passive ferromagnetic counterweight 31c may be fixed to the gantry 12g.

1.-9. (canceled)

10. A medical apparatus comprising:
a magnetic resonance imaging device for acquiring magnetic resonance data from an imaging volume, the magnetic resonance imaging device including:
a main magnetic unit for generating a uniform main magnetic field within the imaging volume,
an RF unit for generating an oscillating electromagnetic field,
one or more gradient coils for generating pulse sequences, the pulse sequences having an excitation portion for exciting spin of nuclei of excitable atoms in a selected slice of the imaging volume during an excitation period and spatial encoding portions for the excited atoms during spatial encoding periods, and
one or more antennas for receiving RF signals emitted by excited atoms;
a particle beam apparatus having a particle beam line for producing and directing a beam of charged particles;
a magnetic correction device for applying a magnetic correction to a magnetic field perturbation within the imaging volume; and
a controller configured to provide, to the magnetic correction device, a spatially optimized magnetic correction within a restricted volume of the imaging volume during a spatially optimized magnetic correction period, the spatially optimized magnetic correction period being posterior to the excitation period and overlapping with at least one spatial encoding period, and the restricted volume includes the selected slice.

11. The medical apparatus of claim 10, wherein the restricted volume is the selected slice.

12. The medical apparatus of claim 10, wherein the restriction volume includes the selected slice and a buffer zone surrounding, at least in part, the selected slice.

13. The medical apparatus of claim 10, wherein the controller is further configured to provide a magnetic correction within the whole imaging volume during a time period preceding the spatially optimized magnetic correction period and overlapping with the excitation period.

14. The medical apparatus of claim 10, wherein the particle beam apparatus comprises at least one bending magnet and at least one scanning magnet for directing the particle beam to an irradiation volume within the imaging volume.

15. The medical apparatus of claim 14, wherein the at least one bending magnet and the at least one scanning magnet are configured to generate an adjustable magnetic field.

16. The medical apparatus of claim 15, wherein the controller is further configured to apply the spatially optimized magnetic correction according to set point information associated with the at least one bending magnet and the at least one scanning magnet.

17. The medical apparatus of claim 10, wherein the particle beam apparatus comprises a gantry configured to rotate around a rotational axis.

18. The medical apparatus of claim 17, wherein the particle beam apparatus further comprises at least one bending magnet and at least one scanning magnet, and wherein the controller is further configured to apply the spatially optimized magnetic correction based on a rotational angle of the gantry.

19. The medical apparatus of claim 10, wherein the controller is further configured to coordinate the pulse sequences with the direction of the beam of charged particles and the spatially optimized magnetic correction.

20. The medical apparatus of claim 10, wherein the magnetic correction device comprises one or more active coils arranged to generate magnetic correction of a longitudinal component and transversal components of the magnetic field perturbation.

21. A method for correcting a magnetic field perturbation within an imaging volume, wherein the magnetic field is generated by a magnetic resonance imaging apparatus, the method comprising:
generating pulse sequences having an excitation portion for exciting spin of nuclei of excitable atoms in a selected slice of the imaging volume generated during an excitation period and having spatial encoding portions generated during spatial encoding periods, and
applying a spatially optimized magnetic correction within a restricted volume of the imaging volume during a spatially optimized magnetic correction period, the spatially optimized magnetic correction period being posterior to the excitation period and overlapping with at least one spatial encoding period, and the restricted volume includes the selected slice.

22. The method of claim 21, wherein the restricted volume is the selected slice.

23. The method of claim 21, wherein the restriction volume includes the selected slice and a buffer zone surrounding, at least in part, the selected slice.

24. The method of claim 21, further comprising applying a magnetic correction within the whole imaging volume during a time period preceding the spatially optimized magnetic correction period and overlapping with the excitation period.

25. The method of claim 21, further comprising providing a particle beam apparatus having at least one bending magnet and at least one scanning magnet for directing a particle beam to an irradiation volume within the imaging volume.

26. The method of claim 25, further comprising:
generating an adjustable magnetic field using the at least one bending magnet and the at least one scanning magnet; and
applying the spatially optimized magnetic correction according to set point information associated with the at least one bending magnet and the at least one scanning magnet.

27. The method of claim 25, wherein the particle beam apparatus further includes a gantry configured to rotate around a rotational axis.

28. The method of claim 27, further comprising applying the spatially optimized magnetic correction based on a rotational angle of the gantry.

29. The method of claim 21, further comprising coordinating the pulse sequences with a direction of a beam of charged particles and the spatially optimized magnetic correction.
